(19) United States
(12) Patent Application Publication
(10) Pub. No.: US 2010/0190192 A1
(43) Pub. Date: Jul. 29, 2010

(54) METHOD OF DIAGNOSING PRE-ECLAMPSIA

(76) Inventor: Victor Voroteliak, Brisbane (AU)

Correspondence Address: EDWARDS ANGELL PALMER & DODGE LLP, P.O. BOX 55874, BOSTON, MA, 02205, US

(21) Appl. No.: 12/311,007

(22) PCT Filed: Sep. 15, 2006
(86) PCT No.: PCT/AU2006/001357

§ 371 Date: Apr. 5, 2010

Related U.S. Application Data
(60) Provisional application No. 60/717,684, filed on Sep. 15, 2005.

(51) Int. Cl. G01N 33/53; C07K 14/00; C07K 16/00; G01N 33/559

(52) U.S. Cl. 435/7.93; 530/350; 530/387.1; 436/501; 204/461; 204/612

(57) ABSTRACT

The present invention relates to a marker for the development of pre-eclampsia. In particular the invention provides a marker for the development of pre-eclampsia, which marker consists of a polypeptide of approximately 26.6 Kd as determined by 15-30% gradient SDS-PAGE under reducing conditions.

### FIELD OF THE INVENTION

The present invention relates to a marker for the development of pre-eclampsia. The present invention also relates to methods of diagnosing and treating pre-eclampsia.

### BACKGROUND OF THE INVENTION

Pre-eclampsia, or pregnancy induced hypertension (PIH) is the most common medical disorder of pregnancy with a reported incidence in the obstetric literature of about 7-10% of all pregnancies (Roberts et al., (1993); In: Fetal Medical Review. Ed. Dunlop, Edward Arnold Publishers, London). The definition/diagnosis of preeclampsia includes elevated blood pressure, proteinuria and edema. Pre-eclampsia is classified as mild or severe, where one or more of the following criteria may indicate severe preeclampsia including:
1. blood pressure, 160 systolic or 110 diastolic when measured on a resting patient on two or more occasions at six hourly intervals,
2. proteinuria is 5 g/24 h
3. urine production is 400 mL/24 h
4. cerebral/visual disturbances,
5. epigastric pain,
6. pulmonary edema,
7. impaired liver function, and
8. thrombocytopenia.

It is also known as pre-eclamptic toxemia and its more severe form, eclampsia, is associated with generalised convulsions or seizures. The incidence ranges between 2-35% depending on the diagnostic criteria used and the population studied (Sibai, (1991), Clin Obstet Gynecol, 34:27-34). The incidence of pre-eclampsia is increased significantly in nulliparous women, in women with a family history of PIH, in women with previous PIH, in diabetic women, and in women whose pregnancies are associated with increased trophoblastic mass (Zhou et al., (1992), J. Clin Invest, 91:950-960).

PIH is one of the major causes of maternal death throughout the world, and in the United States and England and Wales is an important cause of fetal and neonatal morbidity and mortality (Kaunitz et al., (1985), Obstet Gynecol, 65:605-612; Department Of Health: Report on confidential'enquiries into maternal deaths in the United Kingdom. 1985-1987, HMSO. London; 1991). Analysis of a large, mainly hospital-based set of data collected by the World Health Organisation (WHO) indicates that PIH is responsible for 10-15% of the maternal mortality in various developing countries such as Asia, Africa, Latin America and the Caribbean.

Studies have demonstrated that PIH can in some cases be prevented (Uzan et al., (1992), J Gynecol Obstet Reprod, 21:315-318) if treatment is started early in the pregnancy. However, uncertainties about the safety of the interventions, such as low dose aspirin, remain an obstacle to their use on unselected populations (Masse et al., (1993), Am J Obstet Gynecol, 169:501-508; Roberts (1994), J Nurse Midwifery, 39(2):70-90) and thus treatment is usually not started until the blood pressure has already begun to rise. Early identification of women at high risk of developing PIH is not yet reliable. Familial and medical histories fail to identify most individuals who subsequently develop PIH and until now the candidate clinical or laboratory tests which have been used either individually or in combination have also demonstrated poor predictive values (Roberts supra).

Thus Dionne and co-workers in 1994 stated that “[t]he development of new markers, with sufficiently good predictive values to be used as screening tests in the prediction of preeclampsia, would have a major impact on the prevention of this complication of pregnancy” (Clin Biochem, 27(2):99-103).

### Diagnosis of Established PIH

The clinical criteria for the diagnosis of established pregnancy induced hypertension as proposed by the American College of Obstetricians and Gynaecologists (ACOG) are: a systolic blood pressure of >140 mm Hg: a diastolic blood pressure of >90 mm Hg; an increase of >30 mm Hg in systolic pressure; an increase of >15 mm Hg in diastolic pressure when any one of the above mentioned criteria are present on at least two occasions separated by an interval of six hours or longer. The presence of peripheral edema or proteinuria (defined as >300 mg/24 h or, >1 g/L on two or more random urine samples at least six hours apart) are also required for diagnosis. The diagnosis is particularly difficult in women with PIH superimposed on pre-existing chronic hypertensive, vascular or renal disease (Paso & Christianson (1976), Am J Obstet Gynecol, 125:740-745; Sidal (1988), Am J Obstet Gynecol, 159:1-5; Sandoval et al., (1994), Ginecol Obstet Mex, 61:283-289). Oedema and abnormal weight gain are used in the diagnosis of PIH. However oedema occurs in about 80% of pregnancies and generalised edema and excess weight gain are common in normal pregnancy (Dexter & Weiss (1941), Boston. Little Brown: 22; Fadigan et al., (1994), Am Fam Physician, 49:849-856).

The presence of proteinuria is commonly detected by using dipsticks on random urine samples. However, the concentration of protein in random urine samples is highly variable and influenced by several factors such as contamination (false positive result), exercise (increased excretion), low specific gravity (false negative) and high specific gravity (false positive) (Gleicler et al., (1986), Am J Obstet Gynecol, 155:1011-1016; McEwan (1987), In: Hypertension in pregnancy. Sharp & Symonds (eds), Perinatology Press; 63-67). There remains considerable controversy regarding the degree of protein excretion necessary for the diagnosis and the reliability of urinary protein dipsticks (Meyer et al., (1994), Am J Obstet Gynecol, 170:137-141).

### Clinical Criteria for Identifying Individuals at High Risk of Developing PIH

At present, there is no reliable way of predicting which women will develop pre-eclampsia. However, there are groups of pregnant women who are at higher than average risk of developing the disorder. They include nulliparous women (primigravidae), teenagers or women over 35 years of age, women with multiple gestations, women with gestational diabetes, women with a history or evidence of chronic hypertension, and women who were hypertensive during a previous pregnancy. Excluding nulliparae, this group of women has a 25 percent chance of developing pre-eclampsia, but accounts for only 10 percent of cases. Nulliparae account for 60 percent of cases, but these women have only a 1 in 6 chance of developing pre-eclampsia. The remaining 20 percent of cases have no risk factors at all.

The supine pressor test was studied in twelve reports (Sidal supra), the sensitivity in predicting pre-eclampsia ranged from 8-93% and specificity ranged from 54-91%. The false positive rate was as high as 90%. The angiotensin II infusion test (Chesley (1975), J Reprod Med, 15:173-178) had a sensitivity of 90-95% although the sensitivity was highly variable, with a high incidence of false-positive tests. In addition, the test is complex and expensive and is not practical for clinical use. However, the fact that this test gives abnormal results many Weeks before the onset of hypertension indicates that the initial pathological changes of the condition are present many weeks before the development of overt hypertension.

Thus there remains a need to develop and establish a test capable of predicting which women will develop pre-eclampsia.

### SUMMARY OF THE INVENTION

The present inventor found that a novel 26.6 Kd polypeptide was present in the sera of pregnant women presenting with pregnancy-induced hypertension (PIH) as compared to women without PIH. Research showed that women having the 26.6 Kd polypeptide present were at greater risk of developing eclampsia than women without the 26.6 Kd polypeptide.

The inventor further confirmed that the polypeptide was useful as a marker for the development of pre-eclampsia.

Results from further research has also shown that the polypeptide is a marker for pre-eclampsia and a potential target for therapeutic agents directed against the development of pre-eclampsia.

Accordingly, in a first aspect the present invention provides a marker for the development of pre-eclampsia, which marker consists of a polypeptide of approximately 26.6 Kd as determined by 15-30% gradient SDS-PAGE under reducing conditions.

In a second aspect, the present invention provides a method of detection of a marker for the development of pre-eclampsia from a maternal sample taken from a pregnant human, which method comprises determining in the maternal sample the presence of a polypeptide of approximately 26.6 Kd as determined by 15-30% gradient SDS-PAGE under reducing conditions as compared to a polypeptide of approximately 26 Kd found in a sample taken from a human not affected by pre-eclampsia.

In a third aspect the present invention provides a method of diagnosing and/or predicting pre-eclampsia (PE) in a pregnant human, which method comprises detecting in a maternal sample the presence of a polypeptide of approximately 26.6 Kd as determined by 15-30% gradient SDS-PAGE under reducing conditions as compared to a polypeptide of approximately 26 Kd found in a sample taken from a human not affected by pre-eclampsia.

In a fourth aspect the present invention provides a diagnostic kit for the detection of pre-eclampsia (PE) in a pregnant human comprising as a positive control a polypeptide of approximately 26.6 Kd as determined by 15-30% gradient SDS-PAGE under reducing conditions which polypeptide has been isolated from a pregnant human having pre-eclampsia.

In a fifth aspect the present invention provides an antibody capable of selectively binding to a polypeptide of approximately 26.6 Kd as determined by 15-30% gradient SDS-PAGE under reducing conditions which polypeptide has been isolated from a pregnant human having pre-eclampsia.

In a sixth aspect the present invention provides an inhibitor of the development or progression of pre-eclampsia in a pregnant human, wherein said inhibitor is capable of reducing or removing the presence of a polypeptide of approximately 26.6 Kd as determined by 15-30% gradient SDS-PAGE under reducing conditions from the serum of a pregnant human having pre-eclampsia or at risk from developing pre-eclampsia. Preferably, the inhibitor is capable of reducing the level of expression of the 26.6 Kd polypeptide.

In an seventh aspect the present invention provides antibody that is specific to a polypeptide of approximately 26.6 Kd as determined by 15-30% gradient SDS-PAGE under reducing conditions isolated from the serum of a pregnant human having pre-eclampsia.

In a eighth aspect the present invention provides a method for the detection of pre-eclampsia in a mammal, comprising the steps of: 1) obtaining a maternal sample from a mammalian subject; 2) contacting the sample with an antibody for a 26.6 Kd polypeptide marker found in the serum of a woman suffering pre-eclampsia, to allow formation of a complex of the antibody and the 26.6 Kd polypeptide marker; and 3) detecting the antibody-marker complex.

In a ninth aspect the present invention provides a method of monitoring the effectiveness of a treatment for pre-eclampsia comprising the steps of: 1) providing a treatment to a mammalian subject experiencing pre-eclampsia; 2) obtaining at least one post-treatment maternal sample from the subject; and 3) detecting the presence or absence of a 26.6 Kd polypeptide marker for pre-eclampsia in the post-treatment sample.

In a tenth aspect the present invention provides a kit for use in detecting the presence of a 26.6 Kd polypeptide marker for pre-eclampsia in a maternal sample taken from a subject, comprising: 1) a means for acquiring a quantity of a maternal sample; 2) a media having affixed thereto a capture antibody capable of complexing with a 26.6 Kd polypeptide marker for pre-eclampsia; and 3) an assay for the detection of a complex of the 26.6 Kd polypeptide marker for pre-eclampsia and the capture antibody.

In an eleventh aspect the present invention provides a competitive enzyme linked immunosorbent assay (ELISA) kit for determining the pre-eclampsia status of a mammalian subject, comprising a first antibody specific to a 26.6 Kd polypeptide marker for pre-eclampsia to detect its presence in a maternal sample of the subject.

### BRIEF DESCRIPTION OF THE FIGURES

FIG. 1 shows the resolution of semi-purified PIH (P) and normal pregnant (N) serum proteins by sodium dodecyl sulfate-polyacrylamide gel electrophoresis (SDS-PAGE) and visualisation by silver staining. The analyte specific to PIH is shown (arrow). Molecular weights were calculated from protein standards (S) [Biorad] of known size, these were: (A) Phosphorylase B 110 kD; Bovine seum albumin, 84 kD; (C) Ovalbumin, 47 kD; (D) Carbonic Anhydrase (Bovine erythrocytes), 33 kD; (E) Soybean trypsin inhibitor, 24 kD; (F) Lysozyme, 16 kD (Top to Bottom).

FIG. 2 shows the resolution of semi-purified PIH (P) and normal pregnant (N) serum proteins by SDS-PAGE and visualisation by coomassie staining. The analyte specific to PIH is shown (arrow). Molecular weights were calculated from protein standards (S) [Biorad] of known size, these were: (A) Phosphorylase B 110 kD; Bovine seum albumin, 84 kD; (C) Ovalbumin, 47 kD; (D) Carbonic Anhydrase (Bovine erythrocytes), 33 kD; (E) Soybean trypsin inhibitor, 24 kD; (F) Lysozyme, 16 kD (Top to Bottom).

FIG. 3 shows the resolution of semi-purified PIH (P) and normal pregnant (N) serum proteins by SDS-PAGE and visualisation by silver staining. The analyte specific to PIH is shown (arrow). Molecular weights were calculated from protein standards (S) [Biorad] of known molecular weight, these were: (A) Phosphorylase B 110 kD; Bovine seum albumin, 84 kD; (C) Ovalbumin, 47 kD; (D) Carbonic Anhydrase (Bovine erythrocytes), 33 kD; (E) Soybean trypsin inhibitor, 24 kD; (F) Lysozyme, 16 kD (Top to Bottom).

FIG. 4 shows the resolution of semi-purified PIH (P) and normal pregnant (N) serum and women with pregnancy induced hypertension (H) proteins by SDS-PAGE and visualisation by coomassie blue staining. The analyte specific to PIH is shown (arrow). Molecular weights were calculated from protein standards (S) [Biorad] of known size, these were: (A) Phosphorylase B 110 kD; Bovine seum albumin, 84 kD; (C) Ovalbumin, 47 kD; (D) Carbonic Anhydrase (Bovine erythrocytes), 33 kD; (E) Soybean trypsin inhibitor, 24 kD; (F) Lysozyme, 16 kD (Top to Bottom).

FIG. 5 shows the expansion of gel shown in FIG. 3 to highlight the area of the gel containing the analyte of interest. Samples were semi-purified PIH (P) and normal pregnant (N) serum and women with pregnancy induced hypertension (H) proteins by SDS-PAGE and visualisation by coomassie blue staining. The analyte specific to PIH is shown (arrow). Molecular weight markers shown are (E) Soybean trypsin inhibitor, 24 kD; (F) Lysozyme, 16 kD (Top to Bottom).

FIG. 6 shows resolution of semi-purified PIH (P) and normal pregnant (N) serum and women with pregnancy induced hypertension (H) proteins by SDS-PAGE and visualisation by coomassie blue staining. The analyte specific to PIH is shown (arrow). Molecular weights were calculated from protein standards ($) [Biorad] of known size, these were: (A) Phosphorylase B 106 kD; Bovine seum albumin, 80 kD; (C) Ovalbumin, 49.5 kD; (D) Carbonic Anhydrase, 32.5 kD; (E) Soybean trypsin inhibitor, 27.5 kD; (F) Lysozyme, 18.5 kD (Top to Bottom).

### DETAILED DESCRIPTION OF THE INVENTION

Before describing the present invention in detail, it is to be understood that this invention is not limited to particularly exemplified cell culture techniques, serum, media or methods and may, of course, vary. It is also to be understood that the terminology used herein is for the purpose of describing particular embodiments of the invention only, and is not intended to be limiting which will be limited only by the appended claims.

All publications, patents and patent applications cited herein, whether supra or infra, are hereby incorporated by reference in their entirety. However, publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

The practice of the present invention will employ, unless otherwise indicated, conventional techniques of cell biology, cell culture, molecular biology, recombinant DNA, and immunology, which are within the skill of the art. Such techniques are described in the literature. See, for example, Molecular Cloning: A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Methods In Enzymology (Academic Press, Inc., N.Y.); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986).

It must be noted that as used herein and in the appended claims, the singular forms “a,” “an,” and “the” include plural reference unless the context clearly dictates otherwise. Thus, for example, a reference to “a diagnostic sample” includes a plurality of such samples, and a reference to “an antibody” is a reference to one or more antibodies, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

The present invention encompasses the following aspects: preparation of the 26.6 Kd polypeptide of the present invention, which is a marker of the development of pre-eclampsia; preparation of the polynucleotide encoding said polypeptide or a recombinant vector carrying and expressing said polynucleotide; transformants carrying said vector; methods of producing said transformants; antibodies directed against the 26.6 Kd polypeptide of the present invention; methods of detecting the polypeptide; methods of detecting the mRNA or polynucleotide encoding said 26.6 Kd polypeptide; methods of detecting pre-eclampsia; diagnostic kits for the detection of pre-eclampsia; methods of identifying therapeutic agents capable of reducing or removing the presence of the 26.6 Kd polypeptide of the present invention from the serum of pregnant women and methods of treating pre-eclampsia are explained below.

In the description that follows, if there is no instruction, it will be appreciated that techniques such as gene recombinant techniques, production of recombinant polypeptides in animal cells, insect cells, yeast and Escherichia coli, molecular-biological methods, methods of separation and purification of expressed polypeptides, assays and immunological methods, are well-known in this field and any such technique may be adopted.

In its broadest aspect the present invention provides a marker for pre-eclampsia. The term “marker” as used herein refers to the marker for the development of pre-eclampsia (which will also be referred to as pre-eclampsia marker). The marker can be any marker, such as the 26.6 Kd polypeptide described herein, which is present in the serum of a pregnant mammal, preferably human and wherein the mammal is prone to developing pre-eclampsia. An effective pre-eclampsia marker is typically the 26.6 Kd polypeptide described herein; however the marker may also be mRNA encoding the 26.6 Kd polypeptide or a genomic DNA molecule encoding the same. As discussed elsewhere, the 26.6 Kd polypeptide can be isolated from a maternal sample, wherein the polypeptide is approximately 26.6 Kd in size as determined by 15-30% gradient SDS-PAGE under reducing conditions.

In one embodiment, the pre-eclampsia marker consists essential of a 26.6 Kd polypeptide as determined by 15-30% gradient SDS-PAGE under reducing conditions.

The term “maternal sample” as used herein refers to any sample taken from a pregnant, female mammal. Preferably, the mammal is a pregnant human female. Maternal samples that may be analysed by the methods of the present invention can be “taken” i.e. obtained or isolated via swabs, shunts or the like.

Persons skilled in the art will appreciate that the techniques disclosed herein may be used on any type of maternal sample. Preferable the maternal sample is bone marrow, plasma, spinal fluid, lymph fluid, the external sections of the skin from respiratory, intestinal, and genitourinary tracts, tears, saliva, milk, blood; whole blood, serum, blood cells, tumours and organs. Most preferably the maternal sample is serum.

Once taken, the maternal sample may be analysed directly; or may be treated prior to testing by, for example, concentration or pH adjustment. In one preferred embodiment, the maternal sample is serum obtained from blood taken from patients suspected of having pre-eclampsia or at greater risk of developing pre-eclampsia. The serum is initially treated with Affi-Gel Blue gel to remove major interfering compounds (eg. albumin). The detection or quantitation of the pre-eclampsia marker in the maternal sample is then undertaken.

The “detection or quantitation” of a marker of the present invention can be accomplished by any appropriate method including an immunological assay or a molecular-biological assay. When the marker is pre-eclampsia polypeptide, the above method includes, for example, an immunological assay such as Enzyme Linked Immuno Sorbent Assay (ELISA), Radio Immuno Assay (RIA), fluorescence antibody technique, SDS-PAGE, Western blot or an immune structure dyeing method.

When the pre-eclampsia marker is a polynucleotide such as mRNA, the assay includes a molecular-biological assay, for example, Northern blot, Dot blot or polymerase chain reaction (PCR). mRNA can be detected or quantitated by using a pre-eclampsia marker polynucleotide or fragment thereof as a probe or primer.

In one embodiment, the “detection or quantitation” is by SDS-PAGE using a 5 to 20% polyacrylamide gel in accordance with Laemmli, Nature, 227: 680-685, 1970. Preferably, the gradient gels are: 15-23% or 15-25% polyacrylamide gradient gels with a 4% stacking gel. Once the SDS-PAGE gels have run for the required time they are stained with a commercial dye such as 0.25% Coomassie Brilliant Blue R250 (CBB) dissolved in 50% methanol-10% acetic acid to reveal the polypeptide bands. The presence or absence of the 26.6 Kd polypeptide of the present invention can be determined readily by assessing its size against a known standard.

Alternatively, in another preferred embodiment, the “detection or quantitation” of the 26.6 Kd polypeptide of the present invention is by Western Blot or ELISA. As appreciated by those skilled in the art, both of these techniques require the use of an antibody directed to the 26.6 Kd pre-eclampsia polypeptide of the present invention. By using an antibody against the pre-eclampsia polypeptide or fragment thereof, pre-eclampsia can be detected or quantitated.

Both monoclonal and polyclonal antibodies that bind to the 26.6 Kd polypeptide marker of the present invention are useful in the methods and kits of the present invention. The antibodies can be prepared by methods known in the art.

To prepare polyclonal antibody, typically full length 26.6 Kd polypeptide or a part thereof or a polypeptide which includes a part of the 26.6 Kd polypeptide are given as an antigen to a mammal. The polypeptide itself and a carrier, for example, a carrier combined with cattle serum albumin (BSA), keyhole limpet hemocyanin (KLH) or bovine thyroglobulin (BTG) can be used as an antigen. To enhance immune reactions with antigens, for example, complete Freund adjuvants (CFA) and incomplete Freund adjuvants (IFA) can be given. A mouse, a rat, a rabbit, a goat or a hamster can be used as a mammal to immunize. A well known method for producing polyclonal antibodies can be found in Lane et al. (Antibodies: A Laboratory Manual, Second Edition (1989) (Cold Spring Harber Laboratory Press)). Briefly, after the first immunization, a mammal is immunized by an appropriate antigen 3 to 10 times at 1 to 2-week intervals.

A preferable dosage of the antigens is 50 to 100 μg at one time per an animal. When peptides are used, peptides covalently bonded to appropriate carriers are preferably used as antigens. Peptides as antigens can be synthesized by a method of genetic engineering or a peptide synthesizer. Three to seven days after immunization, blood is collected and the responsiveness of the serum against the antigens can be measured by ELISA, see for example, Igaku-Shoin Ltd. (1976), Antibodies: A Laboratory Manual, Second Edition (1989) (Cold Spring Harbor Laboratory Press). Blood is then periodically collected from the immunized mammal until the immunized mammal shows a sufficient antibody titre, and then polyclonal antibodies can be prepared from the serum.

Separation and purification of polyclonal antibodies can be accomplished by chromatography such as a centrifugal separation, salting-out with ammonium sulfate, precipitation with caplyric acid (see, for example, Antibodies: A Laboratory Manual, Second Edition (1989) (Cold Spring Harbor Laboratory Press), DEAE-sepharose column, anion exchange column, protein A column or G-column or a gel filter column.

Once the mammal used to produce polyclonal antibodies has reached an appropriate titre they can also be used to prepare monoclonal antibodies against the 26.6 Kd polypeptide of the present invention. In this procedure spleens or lymph nodes are extracted from the mammal and used to produce hybridoma by fusing an antibody-producing cell from the spleen or lymph node with a myeloma cell. As for the myeloma cell, cells established from a mouse or a rat can be used. Cell fusion can be done according to already known methods, for example, see Kohler and Milstein (1975) (Nature, 256, 495-497).

The 26.6 Kd polypeptide, part thereof or polypeptides including the 26.6 Kd polypeptide or part thereof are injected into a rat. Three to seven days after the rat has shown a sufficient antibody titre, the rat is immunized with the antigen for the last time, and its spleen is extracted as antibody producing cells. The spleen is cut into pieces in MEM medium (Nissui Pharmaceutical Co. Ltd.) and the dissociated cells are precipitated by centrifugation at 1,200 rpm for 5 minutes. Splenocytes are separated by treating the precipitant with Tris-ammonium chloride buffer (pH 7.65) for 1 to 2 minutes to remove red blood cells. The splenocytes are washed with MEM medium 3 times and are used as antibody producing cells.

In order to establish a cell line myeloma cells are isolated from a mouse or rat. Appropriate myeloma cells can be isolated from the following strains: BALB/c (8-azaguanine resistance mouse), P3-X63Ag8-U1 (described as P3-U1) (Current Topics Microbiological Immunology, 81, 1 (1978), SP2/0-Ag14 (described as SP-2) (Nature, 276, 269 (1978).), P3-X63-Ag8653 (described as 653) (Journal of Immunology, 123, 1548 (1979).) or P3-X63-Ag8 (described as X63) (Nature, 256, 495 (1975).). These cell strains are subcultured in a 8-azaguanine medium (a normal medium including 15 μg/ml 8-azaguanine (RPMI1640 medium including 1.5 mM glutamine, 5×10⁻⁵ M 2-mercaptoethanol, 10 μg/ml gentamysin and 10% FCS made by CSL)) and cultured in a normal medium for 3 to 4 days before cell fusion. 2×10⁷ or more cells are prepared for cell fusion.

Hybridoma cells and myeloma cells are then mixed and washed with MEM medium or PBS (per 1 L; 1.83 g sodium phosphate dibasic, 0.21 g monobasic potassium phosphate, 7.65 g NaCl, pH7.2) and mixed as the number of antibody producing cells is 5 to 10 times larger than that of the myeloma cells. After a centrifugal separation at 1,200 rpm for 5 minutes, a precipitant is obtained. The precipitated cells are resuspended in 0.2 to 1 ml of polyethylene glycol solution (2 g polyethylene glycol-1000 (PEG-1000), 2 ml MEM medium, 0.7 ml dimethyl sulfoxide (DMSO)) per 10⁸ antibody producing cells is added to the cells with stirring at 37° C. 1 to 2 ml of MEM medium is then added several times every 1 to 2 minutes. The solution is prepared with MEM medium to 50 ml in total. After a centrifugal separation at 900 rpm for 5 minutes, a precipitant is obtained. 100 ml of HAT medium (normal medium including 10⁻⁴M hypoxanthine, 1.5×10⁻⁵ M thymidine and 4×10⁻⁷M aminopterin) is added to a precipitant and the precipitant is slowly resuspended. The suspension is poured into the 96-well culture plate at 100 μl per well and cultured at 37° C. in the presence of 5% CO₂ for 7 to 14 days.

By the method described in Antibodies: A Laboratory Manual, Second Edition (1989) (Cold Spring Harbor Laboratory Press), hybridomas producing antibodies specifically reacting with 26.6 Kd polypeptide are selected.

Methods of detecting the pre-eclampsia polypeptides or parts thereof using the antibodies described above can involve direct or indirect bonded enzymes, fluorescent substances, radioisotopes or latexes. The assay method, for example, can be ELISA or a chemiluminescence method detecting enzyme activities such as horseradish peroxidase or alkaline phosphatase, FITC method detecting fluorescent tags such as luminol or GFP (Green Fluorescence Protein), RIA method detecting radioisotope tags such as ¹²⁵I or a latex agglutination method detecting binding with latex. The assay can also be, for example, Western blot or immune structure dyeing. Furthermore, the 26.6 Kd polypeptide or a parts thereof can be quantitated by the assay.

The antibodies used in the immunoassays can be immobilized to a solid phase carrier and the trapped polypeptides can be detected by using secondary antibodies with a reporter group or using reagents. Any substance, to which antibodies can attach and which is widely known to persons of ordinary skill in the art, can be used as a solid phase carrier. The substance includes, for example, a microtitre plate, a membrane such as a nitrocellulose membrane, bead, disk, glass, glass fibre, plastic material such as latex, polystyrene or polyvinyl chloride. Magnetic particles or fibre optical sensors (U.S. Pat. No. 5,359,681) can be used.

In this description, “solid phase” means immobilization by a physical method such as adsorption or a chemical binding by a covalent bond between an antibody and a functional group on a carrier. An antibody and a functional group on a carrier can be bonded directly or through a cross-linking agent. Immobilization by a physical method can be accomplished by appropriately diluted antibodies contacted with a carrier, preferably, a microtitre plate or a membrane in an appropriate buffer for an appropriate time. The contact time varies depending on the temperature, but it is typically between about 1 hour and 1 day. About 10 ng to 1 μg, preferably, about 100 to 200 ng of antibodies is added and immobilized on each well of a microtitre plate made of plastic such as polystyrene or polyvinyl chloride. Immobilization by a chemical method can be accomplished by a reaction of a carrier and functional groups of antibodies, for example, a reaction of a carrier and a two-functional reagent that reacts with both hydroxyl groups and amino groups and a carrier. For example, antibodies can be immobilized to a carrier having an appropriate polymer coat with a covalent bond by using benzoquinone or a condensation between aldehyde groups on a carrier and an amine or an active hydrogen on a combination partner.

A carrier-immobilized antibody is treated to inhibit physical adsorption of other polypeptides by a well-known method for a person having ordinary skill in the art with an appropriate blocking reagent, for example, cattle serum albumin or Tween 20 (Sigma-Aldrich). A carrier-immobilized antibody is reacted with a sample and polypeptides of the present invention and antibodies are combined. A maternal sample can be appropriately diluted with an appropriate diluent, for example, phosphate buffered saline solution (PBS). A reaction time of a maternal sample and antibodies should be enough to detect the presence of polypeptides of the present invention in a maternal sample obtained from an individual suspected as having pre-eclampsia, preferably, a time to achieve at least 95% of binding level compared to the level at which bound and not-bound polypeptides are equilibrated.

A time to reach equilibrium can be easily decided by measuring the binding level by the time. Substances other than bound polypeptides can be removed by washing a solid carrier with an appropriate buffer, for example, PBS (including 0.1% Tween 20). Labelled secondary antibodies are reacted with a solid carrier. The labels are preferably enzymes such as horseradish peroxidase, ground substances, supplemental elements, inhibitors, pigments, radioisotopes, colouring substances or fluorescent substances.

The binding between antibodies and labels can be accomplished by well-known methods. The secondary antibodies are reacted for a sufficient time to bind to complexes, which include immobilized antibodies and polypeptides of the present invention. An appropriate time can be easily decided by measuring binding level by the time. The non-binding secondary antibodies can be removed by washing a solid carrier with an appropriate buffer, for example, PBS (including 0.1% Tween 20).

The method of detection of labels of the secondary antibodies depends upon the kind of labels used. For example, when radioisotopes are used as labels, detection by a scintillation counter or an autoradiography can be used. When pigments, colouring substances or fluorescent substances are used as labels, detection by a spectrophotometer can be used. When enzymes are used as labels, substrates for the enzymes are added and reacted for a fixed time and the products are detected by a spectrophotometer. Labels and secondary antibodies can bind directly or indirectly by an avidin-biotin method. When they bind indirectly, one part of the avidin-biotin is bound to a secondary antibody and another is bound to a label. 26.6 kD polypeptide can be detected by a flow through test or a strip test.

In a flow through test, a maternal sample is added to a nitrocellulose membrane on which antibodies are immobilized, and when a sample passes through the membrane, polypeptides bind to the immobilized antibodies to form immune complexes. When a solution including labelled secondary antibodies passes through the membrane, it binds to the immune complexes. In a strip test, once a maternal sample is added, the maternal sample passes through a region including labelled antibodies, and polypeptides bind to labelled antibodies to form immune complexes.

When a maternal sample passes through a region including a solid phase antibody, polypeptides bind to the immune complexes. The quantity of secondary antibodies detected in the region with immobilized antibodies shows the presence or absence of pre-eclampsia.

An alternative to the “detection or quantitation” of the polypeptide of the present invention is the “detection or quantitation” of polynucleotides encoding the pre-eclampsia polypeptide. The polynucleotide encoding the pre-eclampsia marker of the present invention can be used as a marker for pre-eclampsia. The polynucleotide sequence encoding the 26.6 Kd polypeptide of the present invention can be detected and measured using standard molecular-biologically techniques.

One method of detecting the presence of the polynucleotide encoding the 26.6 Kd polypeptide is use of a probe or a primer, which includes nucleotides having the same sequence as the coding sequence of the polypeptide or an oligonucleotide having a sequence complementary to the sequence of the coding sequence of the polypeptide or a derivative thereof. A derivative thereof includes, for example, a oligonucleotide wherein a phosphodiester bond in the oligonucleotide is transformed into a phosphorothioate bond or a N3′-P5′ phosphoamidite bond, a oligonucleotide wherein a ribose and a phosphodiester bond are transformed into a peptide bond, a oligonucleotide wherein a uracil in the oligonucleotide is substituted with a C-5 propionyl uracil or a C-5 thiazole uracil, a oligonucleotide wherein a cytosine in the oligonucleotide is substituted with C-5 propionyl cytosine or cytosine modified with phenoxazine or a oligonucleotide wherein a ribose in DNA is substituted with 2′-O-propyl ribose, 2′-methoxyethoxy ribose or the like. All of the above described polynucleotides are useful, for example, as gene markers, as primers for PCR or as probes for hybridization. The present invention relates to a part or all of the polynucleotides encoding the pre-eclampsia marker of the present invention.

It is also possible that the coding sequence for the 26.6 Kd polypeptide can be isolated, sequenced and/or expressed in vitro. In order to accomplish this a cDNA library, including the polynucleotide encoding the 26.6 Kd polypeptide of present invention, is prepared from human brain, heart, skeletal muscle, spleen, kidney, liver, small intestine, placenta, human normal cells from these tissues or human umbilical vein endothelial cells. A useful method for making cDNA libraries is described in Molecular Cloning: A Laboratory Manual, Second Edition (1989) (Cold Spring Harbor Laboratory Press), Current Protocols in Molecular Biology (1994) (Wiley-Interscience). There are also commercially available kits, for example, SuperScript Plasmid System for cDNA Synthesis and Plasmid Cloning (Invitrogen) or ZAP-cDNA Synthesis Kits (STRATAGENE). Once cDNA including DNA encoding the 26.6 Kd polypeptide of the present invention is obtained it can be inserted into an appropriate expression vector. The expression vector can then be introduced into an appropriate host and transformants obtained.

The expression vector is any vector in which cDNA is inserted and which express in animal cells. Suitable vectors include, for example, pcDNA1.1, pcDNA1.1/Amp, pCDM8, pREP (Invitrogen), pHM6, pHB6 (Roche Diagnostics), pKK223-3, pGEX (Amersham Pharmacia Bioteque), pET-3, pET-11, pBluescriptII SK(+), pBluescriptII SK(−) (STRATAGENE), pUC19, pTrxFus (Invitrogen), pUC118, pSTV28 (TaKaRa), pMAL-c2X (New England BioLabs), pAGE107 (Cytotechnology, 3 (2), 133-140 (1990).; JP1991-22979), pAGE103 (The Journal of Biochemistry, 101 (5), 1307-1310 (1987).), pAMo, pAMoA (The Journal of Biological Chemistry, 268 (30), 22782-22787 (1993).), pAMoPRSA (JP1993-336963) or pAS3-3 (JP1990-227075).

Expression vectors, containing the cDNA encoding the 26.6 Kd polypeptide, are introduced into optional animal cells by any method known in the art. When the host is animal cell the following, non-limiting methods may be used: electroporation (Cytotechnology, (1990), 3, 133-140, calcium phosphate method or lipofection (PNAS, USA, (1987), 84, 7413). Appropriate animal cells include Namalwa (Burkitt lymphoma, ATCC: CRL-1432), HCT-15 (human large bowel cancer cell, ATCC: CCL-225), COS-1 (African green monkey's nephrocyte, ATCC: CRL-1650), COS-7 (African green monkey's nephrocyte, ATCC: CRL-1651) and CHO-K1 (Chinese hamster ovary cell, ATCC: CCL-61).

Transformants of the present invention are cultured by generally known and commonly used methods. It can be accomplished with a medium appropriate to a transforming host and a liquid medium. Examples of useful medium are, MEM medium (Science, 130, 432 (1959).), D-MEM medium (Virology, 8, 396 (1959).), PRMI 1640 medium (The Journal of the American Medical Association, 199, 519 (1967).), YT medium or BEM medium can be used. When transformants are prepared using animal cells as the host the medium is usually supplemented with fetal calf serum (FCS). The medium can optionally also include a substance promoting transcription activity to enhance transcription activity of a promoter of an expression vector. For example, isopropyl-1-thio-[beta]-D-galactopyranosin (IPTG) can be used. The medium might also include others nutrients such as glucose, amino acid, peptone, vitamin, hormone or serum, preferably, FCS, calcium chloride or magnesium chloride.

Alternative methods of obtaining cDNA encoding the polypeptide of the present invention include the chemical synthesis of a polynucleotide sequence or the production of cDNA from extracted mRNA. For example, on the basis of the amino acid sequence of the 26.6 Kd polypeptide of the present invention the polynucleotide sequence can be ascertained. Chemical synthesis of DNA can then be accomplished using a DNA synthesizer by the thiophosphite method (Shimazu Corporation) or using a DNA synthesizer model 392 by the phosphoamidite method (Perkin Elmer, Inc.). cDNA can be prepared from mRNA in cells expressing complementary mRNA of the DNA for the 26.6 Kd polypeptide as a template.

When cDNA encoding the 26.6 Kd polypeptide has been isolated the DNA can be expressed in vitro. For example, polynucleotide coding for the 26.6 Kd polypeptide can be made to express in host cells by subcloning the DNA fragment or a full length DNA downstream of a promoter in an appropriate expression vector. The expression vector is then transformed into a prokaryotic cell, yeast, an animal cell, a plant cell or a insect cell. Appropriate expression vectors include pBTrp2, pBTac1, pBTac2 (Roche Diagnostics), BluescriptII SK(+), pBluescriptII SK(−) (STRATAGENE), pSTV28, pUC118, pUC19 (TaKaRa), pKK233-2 (Pharmacia), pSE280, pSupex, pUB110, pTP5, pC194, pTrxFus (Invitrogen), pGEMEX-1 (Promega), pQE-8 (QIAGEN), pGEX (Pharmacia), pETsystem (Novagen), pMAL-c2 (New England BioLabs), pKYP10 (JP1982-110600), pKYP200 (Agricultural Biological Chemistry, 48, 669 (1984).), pLSA1 (Agricultural Biological Chemistry, 53, 277 (1989).), pGEL1 (Proceedings of the National Academy of Sciences USA, 82, 4306 (1985).), pEG400 (Journal of Bacteriology, 172, 2392 (1990).), pTrs30 (FERM BP-5407), pTrs32 (FERN BP-5408), pGHA2 (FERN BP-400), pGKA2 (FERN BP-6798), pPA1 (JP1987-233798) or pTerm2 (JP1990-22979, U.S. Pat. No. 4,686,191, U.S. Pat. No. 4,939,094, U.S. Pat. No. 5,160,735).

Any promoter which can express in a host cell such as Escherichia coli can be used. For example, it is a promoter from Escherichia coli or a phage such as trp promoter (Ptrp), lac promoter (Plac), PL promoter, PR promoter or PSE promoter, SPO1 promoter, SPO2 promoter or penP promoter.

Host cells include a prokaryote of Escherichia genus, Serratia genus, Bacillus genus, Brevibacterium genus, Corynebacterium genus, Microbacterium genus or Pseudomonas genus. For example, E. coli strains XL1-Blue, XL2-Blue, DH1 strain, MC1000, KY3276, W1485, JM109, HB101, No. 49, W3110, NY49, BL21 (DE3), BL21 (DE3) pLysS, HMS174 (DE3) or HMS174 (DE3) pLysS can be used. Yeast cells that can be used as hosts include S. cerevisiae species of Saccharomyces genus, S. pombe species of Schizosaccharomyces genus, K. lactis species of Kluyveromyces genus, T. pullulans species of Trichosporon genus, S. alluvius species of Schwanniomyces genus or P. pastoris species of Pichia genus.

Any method of introducing the expression vector into a host can be used. For example, electroporation, spheroplast method or a lithium acetate method.

When an animal cell is used as a host, the following expression vectors can be used: pcDNA1/Amp, pcDNA1, pCDM8, pREP4 (Invitrogen), pAGE 107 (Cytotechnology, 3, 133 (1990).), pAGE 103 (The Journal of Biochemistry, 101, 1307 (1987).), pAMo, pAMoA (pAMoPRSA) (The Journal of Biological Chemistry, 268, 22782-22787 (1993).) or pAS3-3 (JP1990-22705). Any promoter that can express in a host can be used as a promoter, for example, a promoter of IE (Immediate-early) gene of human cytomegalovirus (hCMV), an early promoter of SV40, Long Terminal Repeat Promoter of Moloney Murine Leulemia Virus, a promoter of retrovirus, HSP promoter, SR [alpha] promoter or a promoter of metallothionein. An enhancer of IE gene of human CMV can be used with a promoter. An animal cell as a host is, for example, HEK293 (a human fetal nephrocyte, ATCC:CRL-1573), Namalwa (Burkitt lymphoma, ATCC:CRL-1432), HeLa (a cell of carcinoma of uterine cervix, ATCC:CCL-2), HBT5637 (a leukemia cell, JP1987-299), BALL-1 (a leukemia cell) or HCT-15 (a large bowel cancer cell) of an established cell from a human, Sp2/0-Ag14 (a mouse myeloma cell, ATCC:CRL-1581) or NSO (a mouse myeloma cell) of an established cell from a mouse, COS-1 (African green monkey nephrocyte (SV40 transformed cell), ATCC:CRL-1650) or COS-7 (African green monkey nephrocyte (SV40 transformed cell), ATCC:CRL-1651) of an established cell from a monkey, CHO-K1 (Chinese hamster ovary cell, ATCC:CCL-61) or BHK-21 (C-13) (Sicilian hamster kidney cell, ATCC:CCL-10) of an established cell from a hamster, PC12 (an adrenal pheochromocytoma, ATCC:CRL-1721) or YB2/0 (a rat myeloma cell, ATCC:CRL-1662) of an established cell from a rat.

Insect cells can also be used as a host. When an insect cell is used as a host, an expression vector is, for example, pVL1392, pVL1393 or pBlueBacIII (Invitrogen) and a virus for infection is, for example, a Vaculovirus which infects insects of Mamestra brassicoe family; Autographa california nuclear polyhedrosis virus (AcMNPV) Bac-N-Blue DNA. A transformation method of an insect cell is, for example, a method described in Baculovirus Expression Vector: A Laboratory Manual (1992) (W.H. Freeman and Company), Molecular Cloning: A Laboratory Manual, Second Edition (1989) (Cold Spring Harbor Laboratory Press), Current Protocols in Molecular Biology (1994) (Wiley-InterScience) or Biotechnology, 6, 47 (1988). A transfer vector including a target gene and baculovirus DNA for infection to an insect cell are added into a culture and a virus expressing a target gene produced by recombinant infects an insect cell to be expressed a polypeptide.

An insect cell as a host is, for example, an established cell from Spodoptera frugiperda (Mamestra brassicoe) or an established cell from Trichoplusia ni. For example, a cell from S. frugiperda includes Sf9 (ATCC: CRL-1711, an ovary cell) or Sf21 (an ovary cell) and a cell strain from T. ni is, for example, High Five or BTI-TN-5B1-4 (an egg cell, Invitrogen).

Once transformants have been produced and cultured the 26.6 Kd polypeptide of the present invention can be isolated and purified. A useful method of isolation/purification of the 26.6 Kd polypeptide is the method described by Sandler (Methods in Enzymology, 83, 458). When the 26.6 Kd polypeptide is produced and accumulated as dissolved polypeptides, the culture solution can be separated from the cells by, for example, centrifugation. If the 26.6 Kd polypeptide exist in the host cells, the cells are extracted and washed with an appropriate buffer such as STE solution and broken into pieces by ultrasonic waves, French press, Manton Gaulin homogenizer or Dynomill. The resultant material is then separated by centrifugation or filtration.

A method of separation/purification of target proteins from crude material can be accomplished with the combination of all kinds of well-known methods of separation/purification. Well-known methods include, for example, a solvent extraction method, a salting-out method with ammonium sulfate, a dialysis, an sedimentation with an organic solvent, an ultrafiltration method, a gel filtration, all kinds of chromatography such as a diethylaminoethyl (DEAE)-sepharose chromatography, an anion chromatography or an ion exchange chromatography using lysine such as DIAION HPA-75 (Mitsubishi Chemical Corporation), a cation chromatography using lysine such as S-Sepharose FF (Pharmacia), a hydrophobic chromatography or an affinity chromatography such as butylsepharose or all kinds of electrophoresis such as a SDS-polyacrylamide gel electrophoresis or an electro-focussing electrophoresis.

Affinity chromatography can be accomplished by using antibodies against 26.6 Kd polypeptide. When 26.6 Kd polypeptide are produced and accumulated as insoluble polypeptides, cells are separated as mentioned above and broken into pieces by an appropriate method. Then a division including the polypeptides is collected. A collected sample is solubilized with a solubilizer like a surfactant such as sodium lauryl sulfate (SDS) or Sodium N-Dodecanoylsalcosinate (salcosiyl). After the solubilized solution is diluted or dialyzed to the concentration that a solubilizer is not or almost not included and the polypeptide is constructed to a normal stereo structure, a purification sample can be obtained by a method of separation/purification as mentioned above.

The present invention also provides a method and kit for assaying the presence of pre-eclampsia marker present in a maternal sample taken from a mammalian subject suspected of having pre-eclampsia. Early detection of the pre-eclampsia can reduce the time for treatment and reduce the risk of developing clinically significant complications.

A simple point-of-care kit that uses principles similar to the widely-used urine pregnancy testing kits, for the rapid detection of the pre-eclampsia marker will allow the clinician to rapidly diagnose pre-eclampsia, and to rapidly institute proven and effective therapeutic and preventive measures. The use of the kit can represent the standard of care for all patients who are at risk of developing pre-eclampsia.

The methods and kits of the present invention can also provide a means for detecting or monitoring pre-eclampsia including the change in status. Thus, the invention also provides a means for a clinician to monitor the progression of the pre-eclampsia (worsening, improving, or remaining the same) following treatment. Typically, the clinician would establish a protocol of collecting and analysing a quantity of maternal sample from the patient at selected intervals. Typically the sample is obtained intermittently during a prescribed period. The period of time between intermittent sampling may be dictated by the condition of the subject, and can range from a sample each 24 hours to a sample taken continuously, more typically from each 4 hours to each 30 minutes.

Using the methods and techniques described herein, both a qualitative level of the 26.6 Kd polypeptide marker present in the maternal sample can be analysed and estimated, and a quantitative level of 26.6 Kd polypeptide marker present in the sample can be analysed and measured. The clinician would select the qualitative method, the quantitative method, or both, depending upon the status of the patient. Typically, the quantity of sample to be collected is less than 1 millilitre, and more typically less than 10 μl. A typical sample can range from about 1 μl to about 1 ml.

Once an indication of pre-eclampsia has been detected, and intervention and treatment of the condition has commenced, the clinician can employ the method and kit of the invention to monitor the progress of the treatment or intervention. Typically, one or more subsequent post-treatment maternal samples will be taken and analysed for the presence of the 26.6 Kd polypeptide marker as the treatment of the pre-eclampsia continues. The treatment is continued until the presence of the 26.6 Kd polypeptide marker in subsequent post-treatment maternal samples is not detected. As the treatment and intervention ameliorate the condition, the expression of 26.6 Kd polypeptide marker, and its presence in the sample, will be correspondingly reduced. The degree of amelioration will be expressed by a correspondingly reduced level of 26.6 Kd polypeptide marker detected in a sample.

A kit for use in the method typically comprises a media having affixed thereto the capture antibody, whereby the maternal sample is contacted with the media to expose the capture antibody to the 26.6 Kd polypeptide marker contained in the sample. The kit includes an acquiring means that can comprise an implement, such as a spatula or a simple stick, having a surface comprising the media. The acquiring means can also comprise a container for accepting the maternal sample, where the container has a serum-contacting surface that comprises the media. In another typical embodiment, the assay for detecting the complex of the 26.6 Kd polypeptide marker and the antibody can comprise an ELISA, and can be used to quantitate the amount of 26.6 Kd polypeptide marker in a maternal sample. In an alternative embodiment, the acquiring means can comprise an implement comprising a cassette containing the media

A method and kit of the present invention for detecting the 26.6 Kd polypeptide marker can be made by adapting the methods and kits known in the art for the rapid detection of other proteins and ligands in a biological sample. Examples of methods and kits that can be adapted to the present invention are described in U.S. Pat. No. 5,656,503, issued to May et al. on Aug. 12, 1997, U.S. Pat. No. 6,500,627, issued to O'Conner et al. on Dec. 31, 2002, U.S. Pat. No. 4,870,007, issued to Smith-Lewis on Sep. 26, 1989, U.S. Pat. No. 5,273,743, issued to Ahlem et al. on Dec. 28, 1993, and U.S. Pat. No. 4,632,901, issued to Valkers et al. on Dec. 30, 1986, all such references being hereby incorporated by reference.

A rapid one-step method of detecting the polypeptide marker of the present invention can reduce the time for detecting the development of pre-eclampsia. A typical method can comprise the steps of: obtaining a maternal sample from a human suspected of a predisposition to the development of pre-eclampsia; mixing a portion of the sample with a detecting antibody which specifically binds to the 26.6 Kd polypeptide marker, so as to initiate the binding of the detecting antibody to the 26.6 Kd polypeptide marker in the sample; contacting the mixture of sample and detecting antibody with an immobilized capture antibody which specifically binds to the 26.6 Kd polypeptide marker, which capture antibody does not cross-react with the detecting antibody, so as to bind the detecting antibody to the 26.6 Kd polypeptide marker, and the 26.6 Kd polypeptide marker to the capture antibody, to form a detectable complex; removing unbound detecting antibody and any unbound sample from the complex; and detecting the detecting antibody of the complex. The detectable antibody can be labelled with a detectable marker, such as a radioactive label, enzyme, biological dye, magnetic bead, or biotin, as is well known in the art.

In one embodiment, the present invention provides a method of identifying compounds capable of inhibiting the development or progression of pre-eclampsia in a pregnant human.

The invention will now be further described by reference only to the following non-limiting examples. It should be understood, however, that the examples following are illustrative, and should not be taken in any way as a restriction on the generality of the invention described herein. In particular, while the invention is described in detail in relation to the use of serum as the maternal sample this does not preclude the use of other samples such as urine.

### Example 1

### Initial Findings

The original finding was that the blood (plasma or serum) of pregnant women with pregnancy induced hypertension or PIH contains a novel analyte which is absent from the blood of normal pregnant women who do not develop PIH, normal non-pregnant women and male bloods.

The following procedure was utilised. Blood obtained from patients with PIH or from the other control groups (normal pregnant and non-pregnant women and males) were initially treated with Affi-Gel Blue gel for removal of major interfering compounds (eg. albumin). The identification of this unique analyte was performed by electrophoresis using SDS-PAGE. Gels were prepared with a narrow linear gradient range, as the structural difference between the polypeptide band in non-PIH patients and that in the PIH patient varied by 4-6 amino acids. The analyte has been shown to be a possible tetramer by gel permeation chromatography consisting of four subunits, each with a molecular weight of approximately 26.6 Kd compared to the subunit molecular weight of approximately 26 Kd found in non-PIH patients blood.

### Example 2

### Clinical Studies

Blood samples obtained from patients giving a clinical history of pregnancy induced hypertension were retrieved from the routine laboratory and stored at −20° C. Subsequently the placentas were sent for histological examination. We selected for further study, the plasma samples from those patients in whom the diagnosis of PIH was later confirmed by histological examination of the placenta. Positive histological findings confirming the clinical diagnosis of PIH were accelerated maturation of the placenta for the stated period of gestation, and the presence of numerous placental villous infarcts and for intervillous, subchorionic or marginal haemorrhage, necrosis of the decidua basalis and/or haemorrhage with thinning or a lack of maternal blood vasculature. In each case, the histopathological findings were consistent with the clinical impression of PIH.

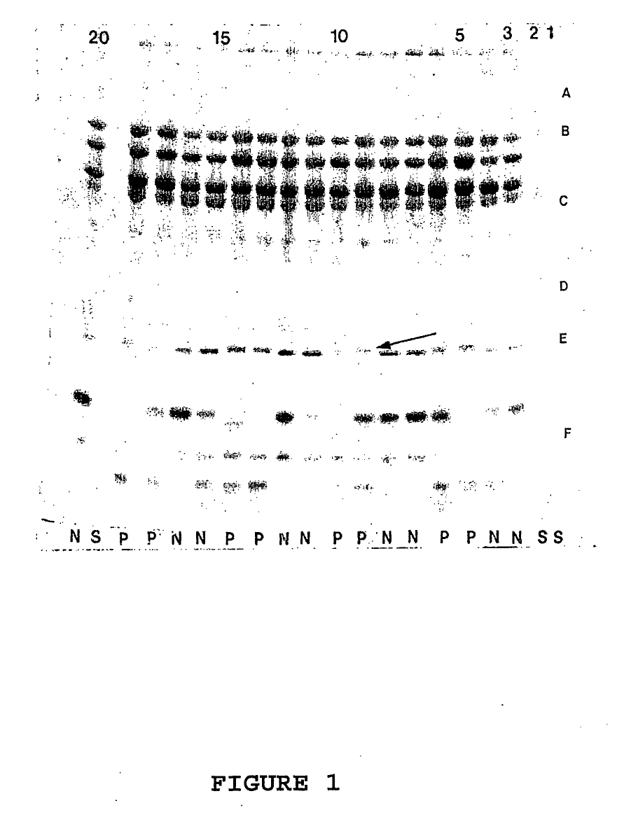

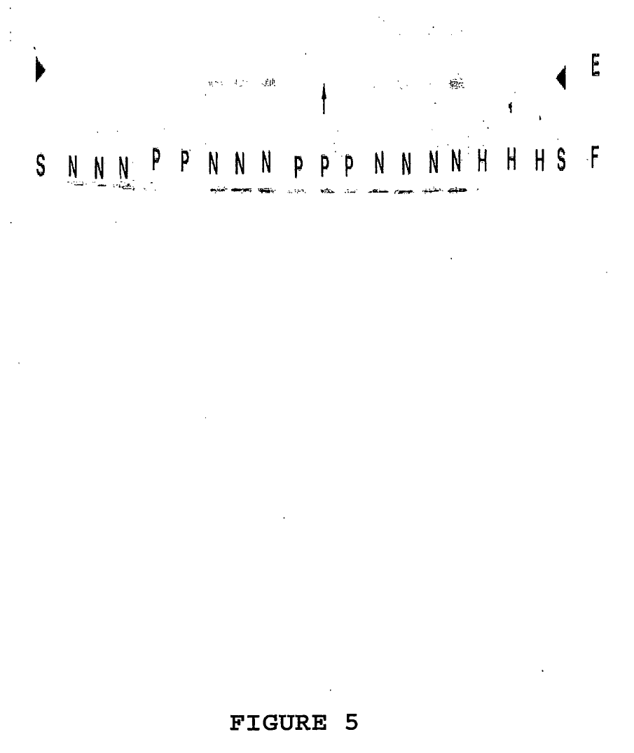

The analyte has been found in the serum of 65 women with PIH who have been studied. It was absent in the blood of 160 women who had normal pregnancies. Results of studies of 30 different PIH, 37 normal pregnant, 7 normal non-pregnant women and 6 males are shown in FIGS. 1-6. The analyte of interest is present in sera or plasma of women with PIH and absent in sera or plasma of all other normal non-PIH groups. This suggests that the analyte is not the result of activation of proteins involved in the coagulation or related cascades.

Bloods from patients were obtained during their second visit (29-34 weeks gestation) after their routine tests had been performed and were used in our clinical trial by examining their protein patterns by gradient SDS-PAGE. Of these, four patients who displayed no signs of high or increased blood pressure and had no abnormal blood chemistries at that time showed the band which was evident in patients with PIH in the electrophoretic study. Later examination of these four patients' histories showed that all four developed high blood pressure between 36-39 weeks. In three of the patients where histological examination of the placenta was performed, the findings were consistent with PIH.

### Example 3

### Properties of the Identified Analyte

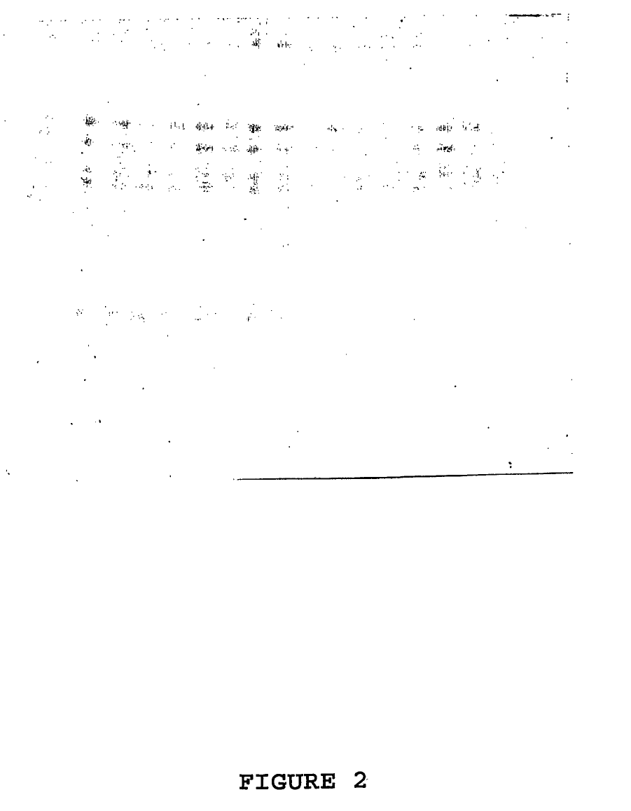

The subunit molecular weight of the analyte found in the blood of women with PIH has been determined by gradient SDS-PAGE to be approximately 26.6 Kd. The term approximately refers to inaccuracies associated with SDS-PAGE; however, the size of the 26.6 Kd polypeptide contrasts with the 26 Kd polypeptide found in the sera of non-PIH subjects. Other inherent structural differences in the amino acid composition of the analyte can be seen from FIGS. 2 and 4, 5; where we have low levels of staining of the analyte with coomassie blue stain relative to the band in non-PIH patients compared with equivalent staining when silver stain is used (FIGS. 1, 3 and 5).

### Example 4

### Electrophoretic Comparison of Semi-Purified Blood from Normal Pregnant Women and Pregnant Women with PIH

Lithium heparin blood samples from patients giving a clinical history of pregnancy induced hypertension were retrieved from the routine laboratory and the plasma stored at −20° C. Subsequently the placentas were sent for histological examination. We selected for further study, the plasma samples from those patients in whom the diagnosis of PIH was later confirmed by histological examination of the placenta. Positive histological findings confirming the clinical diagnosis of PIH were accelerated maturation of the placenta for the stated period of gestation, and the presence of numerous placental villous infarcts with/without intervillous, subchorionic and/or marginal haemorrhage, necrosis of the decidua basalis and/or haemorrhage with thinning or a lack of maternal blood vasculature. In each case, the histopathological findings were consistent with the clinical impression of PIH.

Prior to use, Affi-gel blue gel (AGB [BIORAD]) was washed with 1.4M NaCl in 20 mM phosphate buffer pH 7.1. The gel was then washed a further three times in PBS (NaCl: 8.0 g/L [BDH]; Na₂HPO₄: 1.15 g/L [BDH]; KCl: 0.2 g/L [BDH]; KH₂PO₄.2H₂O: 0.2 g/L [BDH]). Following each wash, the gel was centrifuged at 4500 rpm for 10 min and the supernatant discarded. After the third centrifugation step, the gel was resuspended to its original volume (50 mL) in PBS.

Patient sera (all sera from disease specific and negative (control) groups; 12-20 patients per group) (50-250 μL) were treated with AGB in an exact ratio of 1:5 respectively and mixed end-over-end for 30 min for the removal of albumin and other proteins commonly found in the blood. They were then centrifuged for 10 min at 10,000 rpm and the supernatant retained. All supernatants were applied to a affinity matrix (1 mL) using concavalin-A as the affinity ligand; the matrix was pre-equilibrated with 0.02M Tris buffer pH7.4 containing the following: 0.5M NaCl, 0.1 mM CaCl₂ and 0.1 mM MnCl₂. All samples were applied at a flow rate of 0.5 mL/min and wash fractions collected for further processing. The bound fraction was eluted as a single peak with 50 mM Methyl-D-Glucoside and used for further investigations.

The supernatant (50 μL) was mixed with 0.2 ml electrophoresis sample buffer (20% glycerol (v/v), 2% (w/v) SDS, 5% (v/v) 2-mercaptoethano, 0.00125% (w/v) bromophenol blue and 12.5% (v/v) 0.5M Tris-HCl, pH 6.8), boiled for 10 min at 100° C. and 1 μL applied to a gradient gel; 15-23% and 15-25% polyacrylamide gradient gels with a 4% stacking gel were used. Molecular weight standards prepared in-house were also run on the same gel. Gels were equilibrated with running buffer (125 mM Tris, 0.96M glycine, pH 8.0 containing 0.5% (w/v) SDS).

Electrophoresis was performed on the BIORAD Protean II system. Proteins were allowed to electrophorese at 40 ma constant current for approximately five hours or until the dye front was near the end of the gel. Proteins were visualised using silver stain [BIORAD] or with coomassie blue [SIGMA].

FIGS. 1-5 show the resultant electrophoretic protein patterns of patients with pregnancy induced hypertension (n=25) with normal pregnant women (n=28), normal non-pregnant women (n=7) and normal male (n=6) sera. The protein band of interest has a different migration in PIH to that found in normal samples, and the PIH band on finer separation had shown that this band could be separated to two bands. These results in a step-wise banding pattern which was found to be consistent and characteristic of blood obtained from women with PIH. The subunit molecular weights interpolated from the standard curve were approximately 26.6-26.8 kD and 25.8-26 kD for the PIH and control migrating bands respectively; this can be seen from FIGS. 1-5. This difference may be due to a loss of 4-6 amino-acids in normal pregnant women or may be due to a variation in carbohydrate composition of the protein or proteins. Patients with PIH also displayed a more diffused migrational pattern than normal sera, and it is difficult to confirm whether the PIH band is the result of one, two or multiple protein bands and subsequently the result of one or more proteins.

Similar variations in banding patterns were also observed from both serum and plasma samples from PIH patients when compared electrophoretically with serum and plasma samples from normal patients. This suggests that the different migration pattern in PIH is not the result of activation of proteins involved in the coagulation or related cascades.

Bloods from patients were obtained during their second visit (29-34 weeks gestation) after their routine tests had been performed and were used in our clinical trial by examining their protein patterns by gradient SDS-PAGE. Of these, four patients who displayed no signs of high or increased blood pressure and had no abnormal blood chemistries at that time showed the band which was evident in patients with PIH in the electrophoretic study. Later examination of these four patients histories showed that all four developed high blood pressure between 36-39 weeks. In three of the patients where histological examination of the placenta was performed, the findings were consistent with PIH.

TABLE 1
Sera collected from PIH (P) and normal (N) normal women of
varying gestational ages were tested for the presence of the
analyte by means of SDS-PAGE in the presence of β-
mercaptoethanol. Protein patterns were visualised both with
silver (FIG. 1) and coomassie blue stain (FIG. 2).

| Gel Track | Specimen | | |
|---|---|---|---|
| Number | Number | Gestational Age (K) | Description |
| 1 | | — | Standard |
| 2 | | — | Standard |
| 3 | 1 | 24 | N |
| 4 | 2 | 28 | N |
| 5 | 3 | 23 | P |
| 6 | 4 | 26 | P |
| 7 | 5 | 32 | N |
| 8 | 6 | 30 | N |
| 9 | 7 | 24 | P |
| 10 | 8 | 33 | P |
| 11 | 9 | 34 | N |
| 12 | 10 | 26 | N |
| 13 | 11 | 25 | P |
| 14 | 12 | 27 | P |
| 15 | 13 | 22 | N |
| 16 | 14 | 27 | N |
| 17 | 15 | 29 | P |
| 18 | 16 | 35 | P |
| 19 | | — | Standard |
| 20 | 17 | 36 | N |

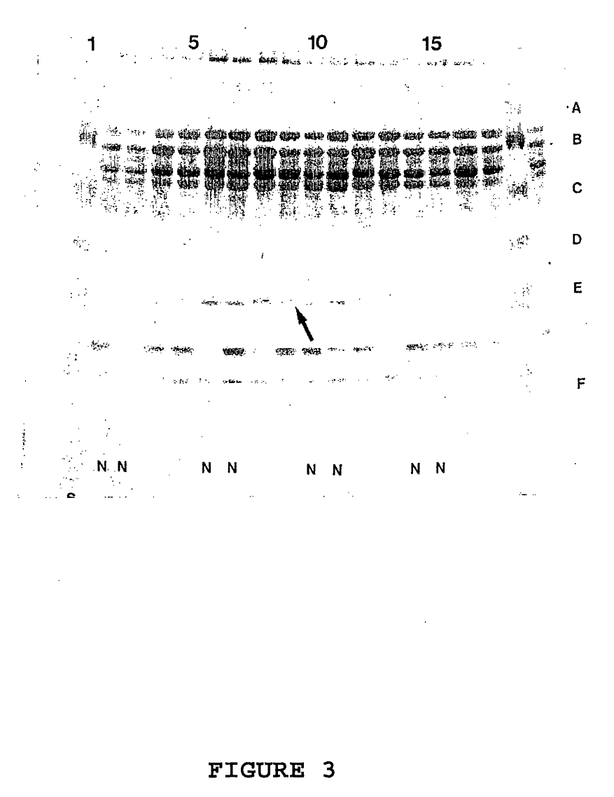

TABLE 2
Further sera collected from PIH (P) and normal (N) pregnant women
of varying gestational ages were tested for the presence of the
analyte by means of SDS-PAGE in the presence of β-
mercaptoethanol. The protein patterns were visualised with
silver stain as shown in FIG. 3.

| Gel Track | Specimen | Gestational | |
|---|---|---|---|
| Number | Number | Age (K) | Description |
| 1 | | — | — |
| 2 | | — | Standard |
| 3 | 18 | 28 | N |
| 4 | 19 | 27 | N |
| 5 | 20 | 26 | P |
| 6 | 21 | 26 | P |
| 7 | 22 | 27 | N |
| 8 | 23 | 30 | N |
| 9 | 24 | 25 | P |
| 10 | 25 | 33 | P |
| 11 | 26 | 34 | N |
| 12 | 27 | 36 | N |
| 13 | 28 | 29 | P |
| 14 | 29 | 30 | P |
| 15 | 30 | 32 | N |
| 16 | 31 | 26 | N |
| 17 | 32 | 34 | P |
| 18 | 33 | 28 | P |
| 19 | | — | Standard |
| 20 | 34 | 32 | N |

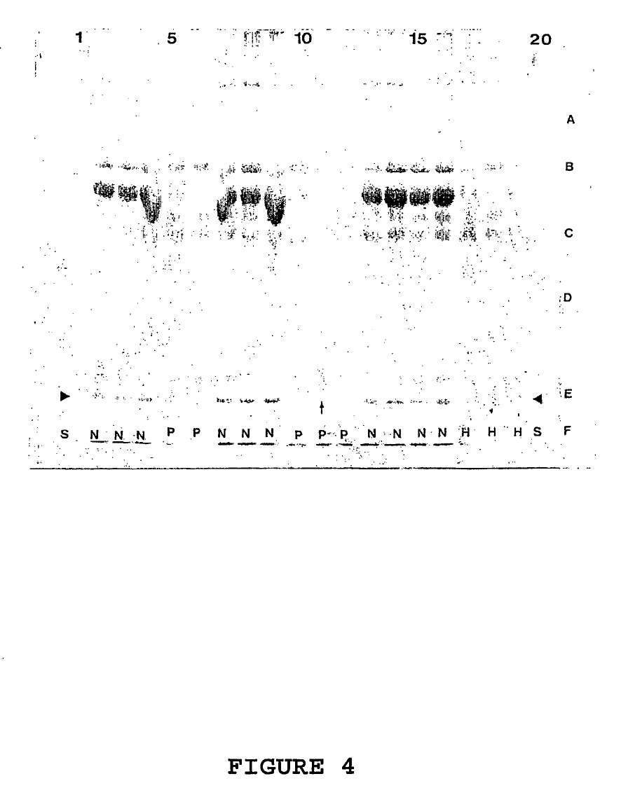

TABLE 3
Further sera collected from PIH (P), normal (N) pregnant women
and women with pregnancy induced hypertension (H) of varying
gestational ages were tested for the presence of the analyte by
means of SDS-PAGE in the presence of β-mercaptoethanol.
Protein patterns were visualised with coomassie blue stain as
shown in FIG. 4.

| Gel Track | Specimen | Gestational | |
|---|---|---|---|
| Number | Number | Age (K) | Description |
| 1 | | — | Standards |
| 2 | 35 | 22 | N |
| 3 | 36 | 24 | N |
| 4 | 37 | 24 | N |
| 5 | 38 | 28 | P |
| 6 | 39 | 29 | P |
| 7 | 40 | 28 | N |
| 8 | 41 | 30 | N |
| 9 | 42 | 32 | N |
| 10 | 43 | 33 | P |
| 11 | 44 | 34 | P |
| 12 | 45 | 29 | P |
| 13 | 46 | 33 | N |
| 14 | 47 | 38 | N |
| 15 | 48 | 40 | N |
| 16 | 49 | 36 | N |
| 17^{□} | 50 | 24 | H |
| 18^{□} | 51 | 23 | H |
| 19^{□} | 52 | 26 | H |
| 20 | | — | Standard |

Notes for Table 3:
Ω: Patient 50 was presented with hypertension, proteinuria was absent.
φ: Patient 51 was presented with hypertension, proteinuria was absent.
δ: Patient 52 was presented with hypertension & proteinuria.

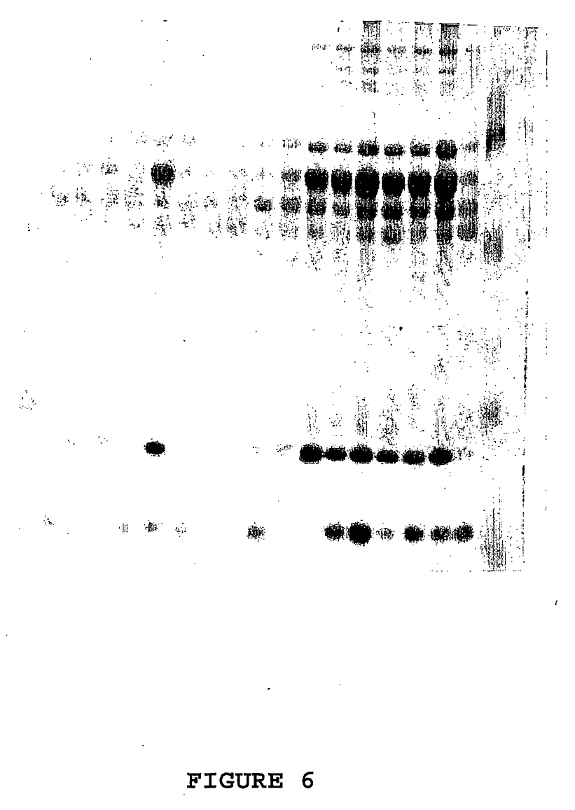

TABLE 4
Further sera collected from PIH (P), normal non-pregnant women
(W) and normal males (M) were tested for the presence of the
analyte by means of SDS-PAGE in the presence of β-
mercaptoethanol. Protein patterns were visualised with coomassie
blue stain as shown in FIG. 6.

| Gel Track | Specimen | Gestational | |
|---|---|---|---|
| Number | Number | Age (K) | Description |
| 1 | | — | Standard |
| 2 | | — | Standard |
| 3 | 53 | — | W |
| 4 | 54 | — | W |
| 5 | 55 | 27 | P |
| 6 | 56 | 29 | P |
| 7 | 57 | — | W |
| 8 | 58 | — | W |
| 9 | 59 | — | W |
| 10 | 60 | — | W |
| 11 | 61 | 33 | P |
| 12 | 62 | 28 | P |
| 13 | 63 | — | M |
| 14 | 64 | — | M |
| 15 | 65 | — | M |
| 16 | 66 | — | M |
| 17 | 67 | — | M |
| 18 | 68 | — | M |
| 19 | 69 | — | W |
| 20 | | — | Standard |

1. A marker for the development of pre-eclampsia, which marker consists of a polypeptide of approximately 26.6 Kd as determined by 15-30% gradient SDS-PAGE under reducing conditions.

2. The marker of claim 1, wherein said marker is present in a maternal sample taken from a pregnant human.

3. A method of detection of a marker for the development of pre-eclampsia from a maternal sample taken from a pregnant human, which method comprises determining in the maternal sample the presence of a polypeptide of approximately 26.6 Kd as determined by 15-30% gradient SDS-PAGE under reducing conditions as compared to a polypeptide of approximately 26 Kd found in a sample taken from a human not affected by pre-eclampsia.

4. A method of diagnosing and/or predicting pre-eclampsia (PE) in a pregnant human, which method comprises detecting in a maternal sample the presence of a polypeptide of approximately 26.6 Kd as determined by 15-30% gradient SDS-PAGE under reducing conditions as compared to a polypeptide of approximately 26 Kd found in a sample taken from a human not affected by pre-eclampsia.

5. A diagnostic kit for the detection of pre-eclampsia (PE) in a pregnant human comprising as a positive control a polypeptide of approximately 26.6 Kd as determined by 15-30% gradient SDS-PAGE under reducing conditions which polypeptide has been isolated from a pregnant human having pre-eclampsia.

6. An antibody capable of selectively binding to a polypeptide of approximately 26.6 Kd as determined by 15-30% gradient SDS-PAGE under reducing conditions which polypeptide has been isolated from a pregnant human having pre-eclampsia.

7. An inhibitor of the development or progression of pre-eclampsia in a pregnant human, wherein said inhibitor is capable of reducing or removing the presence of a polypeptide of approximately 26.6 Kd as determined by 15-30% gradient SDS-PAGE under reducing conditions from the serum of a pregnant human having pre-eclampsia or at risk from developing pre-eclampsia.

8. The inhibitor of claim 7, wherein said inhibitor is capable of reducing the level of expression of the 26.6 Kd polypeptide.

9. A method for the detection of pre-eclampsia in a pregnant human subject, comprising the steps of: 1) obtaining a sample from said subject; 2) contacting the sample with an antibody capable of selectively binding, a 26.6 Kd polypeptide marker found in the serum of a woman suffering pre-eclampsia, to allow formation of a complex of the antibody and the 26.6 Kd polypeptide marker; and 3) detecting the antibody-marker complex.

10. A method of monitoring the effectiveness of a treatment for pre-eclampsia comprising the steps of: 1) providing a treatment to a pregnant human experiencing pre-eclampsia; 2) obtaining at least one post-treatment maternal sample from the human; and 3) detecting the presence or absence of a 26.6 Kd polypeptide marker for pre-eclampsia in the post-treatment sample.

11. A kit for use in detecting the presence of a 26.6 Kd polypeptide marker for pre-eclampsia in a maternal sample taken from a subject, comprising: 1) a means for acquiring a quantity of a maternal sample; 2) a media having affixed thereto a capture antibody capable of complexing with a 26.6 Kd polypeptide marker for pre-eclampsia; and 3) an assay for the detection of a complex of the 26.6 Kd polypeptide marker for pre-eclampsia and the capture antibody.

12. A competitive enzyme linked immunosorbent assay (ELISA) kit for determining the pre-eclampsia status of a mammalian subject, comprising a first antibody specific to a 26.6 Kd polypeptide marker for pre-eclampsia to detect its presence in a maternal sample of the subject.
